(19) United States
(12) Patent Application Publication
(10) Pub. No.: US 2024/0139090 A1
(43) Pub. Date: May 2, 2024

(54) COSMETIC COMPOSITION COMPRISING POLYACRYLATE CROSSPOLYMER-6 AND HIGH HLB NONIONIC SURFACTANT

(71) Applicant: The Procter & Gamble Company, Cincinnati, OH (US)

(72) Inventors: Fanny Himawan, Singapore (SG); Shuhei Tanaka, Singapore (SG)

(21) Appl. No.: 18/081,750

(22) Filed: Dec. 15, 2022

Related U.S. Application Data
(60) Provisional application No. 63/415,643, filed on Oct. 12, 2022.

(51) Int. Cl. A61K 8/81; A61Q 19/08

(52) U.S. Cl. CPC A61K 8/8147; A61Q 19/08; A61K 2800/5422

(57) ABSTRACT

A cosmetic composition containing polyacrylate crosspolymer-6, a nonionic surfactant having HLB of from about 10 to about 20, and an aqueous carrier. The composition meets at least one of the following conditions: (i) the pH of the composition is between about 2.0 and about 5.0; (ii) the nonionic surfactant is liquid at 20° C.; and (iii) the composition is substantially free of hydroxyethylcellulose and xanthan gum. The cosmetic composition can provide improved flowability of the composition.

### FIELD

The present invention relates to a cosmetic composition comprising by weight: (a) from about 1.5% to about 5% of polyacrylate crosspolymer-6; (b) from about 0.05% to about 5% of a nonionic surfactant having HLB of from about 10 to about 20; and (c) aqueous carrier, wherein the composition meets at least one of the following conditions (i)-(iii): (i) wherein the pH of the composition is between about 2.0 and about 5.0; (ii) wherein the nonionic surfactant is liquid at 20° C.; and (iii) wherein the composition is substantially free of hydroxyethylcellulose and xanthan gum. The composition of the present invention provides improved flowability of the composition.

### BACKGROUND OF THE INVENTION

Mammalian keratinous tissue, particularly human skin, is subjected to a variety of insults by both extrinsic and intrinsic factors. Such extrinsic factors include ultraviolet radiation, environmental pollution, wind, heat, infrared radiation, low humidity, harsh surfactants, abrasives, etc. Intrinsic factors, on the other hand, include chronological aging and other biochemical changes from within the skin. Whether extrinsic or intrinsic, these factors result in visible signs of skin damage. Typical skin damages in aging or damaged skin include fine lines, wrinkling, hyperpigmentation, sallowness, sagging, dark under-eye circles, puffy eyes, enlarged pores, diminished rate of turnover, and abnormal desquamation or exfoliation. Additional damage incurred as a result of both external and internal factors includes visible dead skin i.e., flaking, scaling, dryness, and roughness.

Currently, there are a number of personal care products that are available to consumers, which are directed toward improving the health and physical appearance of keratinous tissues such as the skin, hair, and nails. The majority of these products are directed to delaying, minimizing or even eliminating skin wrinkling, spots, and other histological changes typically associated with the aging of skin or environmental damage to human skin. Consumers prefer topically applied products since they are not only effective, but also safe and pleasant to use.

A variety of hydrophilic thickeners are used in such products so that the product has a suitable viscosity, for example, for application to skin.

However, it has been found by the present inventors that, among such hydrophilic thickeners, polyacrylate crosspolymer-6 may provide reduced flowability of the composition, for example, difficulty to pick up a right amount from the package and/or difficulty to see smooth flowability when tilted/shaken which provides very lumpy feel/appearance, especially at a higher level.

Based on the foregoing, there is a need for a cosmetic composition, which provides improved flowability of the composition, while containing a higher level of polyacrylate crosspolymer-6.

### SUMMARY

The present invention is directed to a cosmetic composition comprising by weight:
(a) from about 1.5% to about 5% of polyacrylate crosspolymer-6;
(b) from about 0.05% to about 5% of a nonionic surfactant having HLB of from about 10 to about 20; and
(c) aqueous carrier
   wherein the composition meets at least one of the following conditions (i)-(iii):
(i) wherein the pH of the composition is between about 2.0 and about 5.0;
(ii) wherein the nonionic surfactant is liquid at 20° C.; and
(iii) wherein the composition is substantially free of hydroxyethylcellulose and xanthan gum.

A cosmetic composition comprising by weight:
(a) from about 1.5% to about 5% of polyacrylate crosspolymer-6;
(b) from about 0.05% to about 5% of a nonionic surfactant comprising an HLB of from about 10 to about 20; and
(c) an aqueous carrier;
   wherein the pH of the composition is between about 2.0 and about 5.0;
   wherein the nonionic surfactant is liquid at 20° C.; and wherein the composition is substantially free of hydroxyethylcellulose and xanthan gum.

The composition can provides improved flowability of the composition, while containing a higher level of polyacrylate crosspolymer-6. It has been surprisingly found that, by the use of higher HLB nonionic surfactant, the composition can provide improved flowability compared to compositions having similar viscosities but using a lower HLB nonionic surfactants.

### DETAILED DESCRIPTION

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments, all ingredient percentages are based on the weight of the cosmetic composition, unless specifically stated otherwise. All ratios are weight ratios, unless specifically stated otherwise. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive and combinable to form narrower ranges not explicitly disclosed. For example, delineated upper and lower range limits are interchangeable to create further ranges.

The composition can comprise, consist essentially of, or consist of, the essential components as well as optional ingredients described herein. As used herein, “consisting essentially of” means that the composition or component may only include additional ingredients that do not materially alter the basic and novel characteristics of the claimed composition or method. As used in the description and the appended claims, the singular forms “a”, “an”, and “the” are intended to include the plural forms as well, unless the context clearly indicates otherwise.

### Definitions

“About” modifies a particular value by referring to a range equal to plus or minus twenty percent (+/−20%) or less (e.g., less than 15%, 10%, or even less than 5%) of the stated value.

“Apply” or “application”, as used in reference to a composition, means to apply or spread the compositions of the present invention onto a human skin surface such as the epidermis.

“Derivative,” herein, means amide, ether, ester, amino, carboxyl, acetyl, and/or alcohol derivatives of a given compound.

“Effective amount” means an amount of a compound or composition sufficient to significantly induce a positive benefit to keratinous tissue over the course of a treatment period. The positive benefit may be a health, appearance, and/or feel benefit, including, independently or in combination, the benefits disclosed herein.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration, food additives, and materials used in non-cosmetic consumer products including over-the-counter medications.

“Cosmetic composition” means a composition comprising a cosmetic agent. Examples of cosmetic compositions include color cosmetics (e.g., foundations, lipsticks, concealers, and mascaras), skin care compositions (e.g., moisturizers and sunscreens), personal care compositions (e.g., rinse-off and leave on body washes and soaps), hair care compositions (e.g., shampoos and conditioners).

“Skin care” means regulating and/or improving a skin condition (e.g., skin health, appearance, or texture/feel). Some nonlimiting examples of improving a skin condition include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

“Skin care composition” means a composition that includes a skin care active and regulates and/or improves skin condition.

“Synergy,” and variations thereof, means that the effect provided by a combination of two or more materials is more than the additive effect expected for these materials.

“Treatment period,” as used herein, means the length of time and/or frequency that a material or composition is applied to a target skin surface.

### Cosmetic Composition

The cosmetic composition comprises: polyacrylate crosspolymer-6; a nonionic surfactant having HLB of from about 10 to about 20, wherein the composition meets at least one of the following conditions (i)-(iii): (i) wherein the pH of the composition is between about 2.0 and about 5.0; (ii) wherein the nonionic surfactant is liquid at 20° C.; and (iii) wherein the composition is substantially free of hydroxyethylcellulose and xanthan gum.

It is preferred in the composition that the weight ratio between polyacrylate crosspolymer-6 and the nonionic surfactant is from about 15:1 to about 1:15, alternatively from about 8:1 to about 1:8, alternatively from about 5:1 to about 1:5, alternatively from about 5:1 to about 1:3, in view of providing improved flowability of the composition.

The composition can have a viscosity from about 100 cP to about 60,000 cP, alternatively from about 1000 cP to about 55,000 cP, alternatively from about 1,000 cP to about 48,000 cP, especially when the composition has a higher level of water.

If the composition contains solid oils (having a melting point of 30° C. or more) such as shea butter, cetyl alcohol, stearyl alcohol and/or behenyl alcohol, the composition may contain a limited amount of such solid oils, for providing fresh/light feel from the composition containing a higher level of water. Total level of solid oils in the composition may be up to about 0.5%, alternatively up to about 0.3%, alternatively up to about 0.1%, alternatively 0%.

The composition can be substantially free of hydroxyethylcellulose and xanthan gum, in view of improved flowability by the use of higher HLB nonionic surfactant in the composition containing a higher level of polyacrylate crosspolymer-6. Total level of hydroxyethylcellulose and xanthan gum in the composition can be up to about 0.1%, alternatively up to about 0.05%, alternatively up to about 0.01%, alternatively 0%.

The cosmetic compositions herein are intended for topical application to human skin. The compositions herein may optionally include one or more additional skin actives or other ingredients of the type commonly included in topical cosmetic compositions.

The cosmetic compositions herein may be cosmetic compositions, pharmaceutical compositions, or cosmeceutical compositions, and may be provided in various product forms, including, but not limited to, solutions, suspensions, lotions, gels, toners, cleansing liquid washes hydrogels, film-forming products, and the like. In some instances, the composition form may follow from the particular dermatologically acceptable carrier chosen. For example, the composition (and carrier) may be provided in the form of an emulsion (e.g., water-in-oil, oil-in-water, or water-in-oil-in water) or an aqueous dispersion. The cosmetic composition invention can be in the form of an oil-in-water emulsion.

### pH of the Composition

Typically, cosmetic compositions are formulated to have a slightly acidic to neutral pH (i.e., 5.0-7.0), which is believed to improve the stability of certain ingredients in the composition (e.g., niacinamide, salicylates, and neutralized thickeners). However, formulating a skin care composition at low pH (e.g., 2.0-5.0) may also provide certain benefits such as improving the appearance of skin, bolstering the acid mantle of the skin, exfoliating the skin, improving skin texture, and/or providing flexibility in product formulation.

The composition can have a lower pH, i.e., pH of from about 2.0 to about 5.0, alternatively from about 2.0 to about 4.5, alternatively from about 2.5 to about 4.0, or alternatively from about 3.5 to about 3.9.

It has been found that polyacrylate crosspolymer-6 can provide suitable tolerance to low pH environments and the desired feel and opacity properties to the composition. It has also been found that, at further lower pH, it is preferred to use increased amount of polyacrylate crosspolymer-6 for providing a certain viscosity to the composition, however, such increased amount of polyacrylate crosspolymer-6 tend to cause reduced flowability of the composition especially when the composition has a lower pH. By the use of the above specific nonionic surfactant, the composition provides improved flowability, while having sufficient viscosity coming from polyacrylate crosspolymer-6.

### Low pH Acid Buffering System

A variety of acids are known for use in skin care compositions. For example, alpha hydroxy acids (e.g., citric acid, glycolic acid, malic acid, and lactic acid), beta hydroxy acids (e.g., salicylic acid and propanoic acid), and polyhydroxy acids (e.g., gluconic acid) are commonly used as exfoliants. However, some acids are stronger than others and/or some people may be more sensitive to certain concentrations of acids than others. Both of these factors can increase the risk of skin irritation caused by a low pH composition containing an acid. Lactic acid is one of the gentler alpha hydroxy acids when it comes to skin irritation, but it is still strong enough to provide the desired low pH in the present composition. In addition, lactic acid may provide skin benefits that are not provided by other alpha hydroxy acids (e.g., glycolic acid, citric acid or malic acid). For example, lactic acid may help improve the skin's natural moisture factor and/or stimulate collagen renewal to help improve the signs of aging skin. Thus, the compositions herein include lactic acid at an amount and concentration to provide the skin care composition herein with the desire low pH. In some instances, the low pH composition herein may include 0.5% to 5% lactic acid and/or gluconic acid (e.g., 0.75% to 4%, 1% to 3%, or 1.5% to 2.5%).

When providing a low pH composition for topical application to skin, it is important to include a buffering agent to help maintain the pH of the composition after it is applied to the skin. On average, human skin pH typically ranges from about 5.0 to 6.0. To maintain this pH, human skin has evolved a natural buffering system that resists changes to pH. Thus, when a low pH composition is applied to the skin, the skin's natural buffering system will try to adjust the pH of the composition to match the natural pH of the skin. Without the addition of the buffering agent, the low pH composition may not be able to provide the desired skin care benefit.

The low pH composition herein includes a sodium lactate and/or sodium gluconate buffering agent, depending on the acid(s) used in the composition for lowering the pH. The sodium lactate and/or sodium gluconate buffer may be present at any amount suitable for maintaining the pH of the present composition at the desired level upon application to the skin and for at least 1 minute thereafter (e.g., 5, 10, 15, 30, 60 or even 120 minutes or more after application) in order to provide enough time for the active ingredients in the composition to penetrate into the skin. In some instances, the sodium lactate may be present in the low pH composition at 0.25% to 4% (e.g., 0.5% to 3%, 0.75% to 2% or 1% to 1.75%). In some instances, the salt buffer may be present at a weight ratio acid to buffer of 1:10 to 10:1. It may be desirable to use the L-enantiomer form of the acid and/or salt buffer, since it is the form that occurs naturally in the body. In addition to acting as a buffering agent, sodium lactate may also act as a humectant to help moisturize the skin, which makes it a particularly suitable buffer. Of course, it is to be appreciated that the present composition may optionally include other pH buffers known for use in skin care compositions.

### Polyacrylate Crosspolymer-6

The composition can comprise polyacrylate crosspolymer-6 at a level of from about 1.5% to about 5%, alternatively from about 1.8% to about 4%, alternatively from about 2.0% to about 4.0%.

Polyacrylate crosspolymer-6 is commercially available, for example, as SEPIMAX ZEN from Seppic, France.

### Nonionic Surfactant

The composition can comprise a nonionic surfactant at a level of from about 0.05% to about 5%, alternatively from about 0.1% to about 3%. The nonionic surfactants useful herein have an HLB of from about 10, alternatively from about 11 to about 20, in view of providing improved flowability of the composition. Among them, more preferred are those being liquid at 20° C., in view of, for example, manufacturing easiness. Highly preferred liquid nonionic emulsifiers useful herein include, for example, Glycereth-25 PCA Isostearate having an HLB of 14, and polysorbate-20 having an HLB of 16.7.

Any nonionic emulsifiers can be used as long as the nonionic surfactants have the above HLB. Such nonionic emulsifiers that may be suitable for use herein include ethers of polyglycols and of fatty alcohols, esters of polyglycols and of fatty acids, ethers of polyglycols and of fatty alcohols which are glycosylated, esters of polyglycols and of fatty acids which are glycosylated, ethers of C12-30 alcohols and of glycerol or of polyglycerol, esters of C12-30 fatty acids and of glycerol or of polyglycerol, ethers of oxyalkylene-modified C12-30 alcohols and of glycerol or polyglycerol, ethers of C1-230 fatty alcohols comprising and of sucrose or of glucose, esters of sucrose and of C1230 fatty acids, esters of pentaerythritol and of C12-30 fatty acids, esters of sorbitol and/or of sorbitan and of C12 30 fatty acids, ethers of sorbitol and/or of sorbitan and of alkoxylated sorbitan, ethers of polyglycols and of cholesterol, esters of C12-30 fatty acids and of alkoxylated ethers of sorbitol and/or sorbitan, and combinations thereof. A particularly useful class of emulsifiers is polyethylene glycol ethers of lauryl alcohol such as laureth-1 through laureth-50 (e.g., laureth-4). Still other examples of emulsifiers include ethers of glycerol, polyglycerol, sucrose, glucose, or sorbitol; esters of glycerol, polyglycerol, sucrose, glucose, or sorbitol; and mixtures thereof. Other particularly useful classes of emulsifiers are the alkyl esters of sorbitol and sorbitol anhydrides such as polysorbate 20, polysorbate 21, and polysorbate 40.

Silicone emulsifiers may be suitable for use herein. Linear or branched type silicone emulsifiers may also be used. Particularly useful silicone emulsifiers include polyether modified silicones such as KF-6011, KF-6012, KF-6013, KF-6015, KF-6015, KF-6017, KF-6043, KF-6028, and KF-6038 and polyglycerolated linear or branched siloxane emulsifiers such as KF-6100, KF-6104, and KF-6105; all from Shin-Etsu.

### Silica

The composition can comprise a silica at a level of from about 0.1% to about 10%, alternatively from about 0.3% to about 7%, alternatively from about 0.5% to about 5%.

The silica useful herein can have an average particle size of from about 0.5 microns to about 35 microns, alternatively from about 1 micron to 31 microns, alternatively from about 1 microns to 15 microns, alternatively from about 1 microns to about 10 microns, in view of improved sensory feeling, as too small particles may not provide enough smoothness and too big particles may provide scrubbing-like feeling. The silica can have a spherical shape.

The silica useful herein has an oil absorbance of from about 1 ml/100 g to about 130 ml/100 g, alternatively from about 1 ml/100 g to about 120 ml/100 g, alternatively from about 1 ml/100 g to about 100 ml/100 g, alternatively from about 1 ml/100 g to about 50 ml/100 g, in view of providing a balance between improved sensory feel and moisturized feel.

Commercial examples of spherical silicas having such particle size and such oil absorbance are, for example, Goddball G6C having a mean particle size of 3-5 microns and an oil absorbance of 30 ml/100 g, and Goddball E90C having a mean particle size of 30 microns and an oil absorbance of 120 ml/100 g.

### Lauroyl Lysine

The composition can comprise lauroyl lysine at a level of from about 0.1% to about 10%, alternatively from about 0.3% to about 7%, alternatively from about 0.5% to about 5%.

The composition can comprise lauroyl lysine at a level of from about 0.1% to about 10%, alternatively from about 0.3% to about 7%, alternatively from about 0.5% to about 5%. The shape of the lauroyl lysine can be anything. The shape of the lauroyl lysine can be spherical or flat polygonal (selected from flat pentagonal, flat hexagonal, and flat heptagonal, preferably flat hexagonal shape). Alternatively, the shape of the lauroyl lysine can be flat polygonal.

The lauroyl lysine in the flat polygonal shape can have a mean particle size of from about 10 microns to about 40 microns, alternatively from about 15 microns to about 35 microns, alternatively from about 20 microns to 30 microns, in view of improved sensory feeling, as too small particles may not provide enough bouncy/soft feel and too big particles may provide scrubbing-like feeling.

Commercial example of such lauroyl lysine in the flat polygonal shape having such particle size is, for example, Amihope LL available from Ajinomoto, having a flat hexagonal shape and having a mean particle size of 20-30 microns.

It is preferred that the lauroyl lysine in spherical shape has a mean particle size of from about 1 micron to about 30 microns, alternatively from about 2 microns to about 20 microns, alternatively from about 3 microns to 8 microns.

Lauroyl lysine, especially when it's in flat polygonal shape, may be dispersed in the composition as a layered structure comprising some or several flat polygonal crystals.

It is believed that the lauroyl lysine provides improved moisturizing/cushioning feel, compared to starch solid powders, cellulose solid powders.

Also, compared to hydrophilic solid polymeric powders such as starch solid powders and cellulose solid powders, it is believed that the lauroyl lysine provides reduced pilling and/or clumping especially when the composition contains higher levels of hydrophilic thickeners. For such reduced pilling and/or clumping, it may be preferred that the composition is substantially free of such hydrophilic solid polymeric powders, i.e., contains 0.1% or less, alternatively 0.05% or less of such hydrophilic solid polymeric powders. alternatively, the composition can be free of such hydrophilic solid polymeric powders, i.e., contains 0% of such hydrophilic solid polymeric powders.

It is also believed the lauroyl lysine may provide matt appearance and/or reduced visibility of skin pores.

### Substantially Free of Microplastic Solid Particulates

The composition, when containing silica and/or lauroyl lysine, can provide reduced sticky feel while having sufficient viscosity coming from polyacrylate crosspolymer-6, without the use of microplastic solid particulates. Thus, the composition o, when containing silica and/or lauroyl lysine, may also provide environmental benefit in view of the reduction of the use of microplastic solid particulates. Such microplastic solid particulates are, for example, nylon powder, polyurethane powder, polyethylene powder, silicone resin powder. In the present invention, when containing silica and/or lauroyl lysine, it is preferred that the composition is substantially free of such microplastic solid particulates. alternatively, the composition can be free of such microplastic solid particulates, i.e., contains 0% of such microplastic solid particulates.

### Aqueous Carrier/Water

The composition can comprise an aqueous carrier. The aqueous carrier can be substantially water. Deionized water can be used. Water from natural sources including mineral cations can also be used, depending on the desired characteristic of the composition.

The composition can comprise water at a level of from about 55% to about 99%, alternatively from about 60% to about 98%, alternatively from about 60% to about 95%, alternatively from about 60% to about 90%, by weight of the composition.

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more volatile and/or non-volatile oils (e.g., botanical oils, silicone oils, and/or hydrocarbon oils).

### Skin Conditioning Agent

Optionally, the composition can further comprise a skin conditioning agent. These agents may be selected from humectants and emollients. The amount of skin-condition agent may range from about 1% to about 50%, alternatively from about 2% to about 40%, alternatively from about 5% to about 30%, by weight of the composition.

Humectants are polyhydric alcohols intended for moisturizing, reducing scaling and stimulating removal of built-up scale from the skin. Typical polyhydric alcohols include polyalkylene glycols and alternatively alkylene polyols and their derivatives. Illustrative are propylene glycol, dipropylene glycol, polypropylene glycol, polyethylene glycol, sorbitol, hydroxypropyl sorbitol, hexylene glycol, 1,3-butylene glycol, 1,2,6-hexanetriol, ethoxylated glycerin, propoxylated glycerin and mixtures thereof. The humectant can be glycerin.

When the conditioning agent is an emollient it may be selected from hydrocarbons, fatty acids, fatty alcohols and esters.

### Fatty Alcohol

The compositions herein may include a fatty alcohol. Fatty alcohols refer to high-molecular-weight, straight-chain primary alcohols that have the general structure:

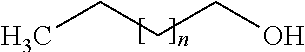

where n=8 to 32.

Fatty alcohols may be natural or synthetic, saturated or unsaturated, branched or straight-chain. Some nonlimiting examples of fatty alcohols commonly used in cosmetic compositions include caprylic, capryl, lauryl, myristyl, cetyl, stearyl, and behenyl alcohols. The fatty alcohols herein may be referred to generically by the number of carbon atoms in the molecule. For example, a “C12 alcohol” refers to an alcohol that has 12 carbon atoms in its chain (i.e., dodecanol).

The fatty alcohol may be included in the compositions herein at 0.0001% to 15% (e.g., 0.0002% to 10%, 0.001% to 15%, 0.025% to 10%, 0.05% to 7%, 0.05% to 5%, or even 0.1% to 3%) by weight of the composition.

### Whitening Agents

The present compositions may contain a whitening agent. The whitening agent useful herein refers to active ingredients that not only alter the appearance of the skin, but further improve hyperpigmentation as compared to pre-treatment. Useful whitening agents useful herein include ascorbic acid compounds, vitamin B 3 compounds, azelaic acid, butyl hydroxy anisole, gallic acid and its derivatives, hydroquinoine, kojic acid, arbutin, mulberry extract, tetrahydrocurcumin, and mixtures thereof. Use of combinations of whitening agents is also believed to be advantageous in that they may provide whitening benefit through different mechanisms.

When used, the compositions can contain from about 0.1% to about 10%, alternatively from about 0.2% to about 5%, by weight of the composition, of a whitening agent.

Ascorbic acid compounds are useful whitening agents and ascorbyl glucoside is a preferred derivative of the ascorbic acid compounds.

### Conditioning Agents

The compositions herein may include 0.1% to 50% by weight of a conditioning agent (e.g., 0.5% to 30%, 1% to 20%, or even 2% to 15%). Adding a conditioning agent can help provide the composition with desirable feel properties (e.g., a silky, lubricious feel upon application). Some non-limiting examples of conditioning agents include, hydrocarbon oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, beeswax derivatives, sterols and phospholipids, salts, isomers and derivatives thereof, and combinations thereof.

Particularly suitable examples of conditioning agents include non-volatile silicone fluids, such as dimethicone copolyol, dimethylpolysiloxane, diethylpolysiloxane, mixed C1-30 alkyl polysiloxanes, phenyl dimethicone, dimethiconol, dimethicone, dimethiconol, silicone crosspolymers, and combinations thereof. Dimethicone may be especially suitable, since some consumers associate the feel properties provided by certain dimethicone fluids with good moisturization.

### Hydrophilic Thickener

In some examples, the compositions may further include additional hydrophilic thickener. When present, the composition can includes from about 0.01% to about 5%, alternatively from about 0.1% to about 4%, and alternatively from about 0.1% to about 3%, by weight of the composition of the hydrophilic thickeners.

The hydrophilic thickener useful herein is not particularly limited as long as it is one that is normally used in cosmetic products. Examples include natural or semi-synthetic water-soluble polymers, synthetic water-soluble polymers and inorganic water-soluble polymers.

As the natural or semi-synthetic water-soluble polymers, polysaccharides and derivatives thereof (including water-soluble alkyl-substituted polysaccharide derivatives) can be used. Specific examples include plant-based polymers such as gum arabic, tragacanth gum, galaetan, guar gum, carob gum, karaya gum, carrageenan, pectin, agar, quince seed (marmelo), algecolloid (phaeophyceae extract), starch (rice, corn, potato, wheat) and glycyrrhizinic acid; microbe-based polymers such as xanthan gum, dextran, succinoglycan and pullulan; starch-based polymers such as carboxymethyl starch and methylhydroxypropyl starch; cellulose-based polymers such as methyl cellulose, nitrocellulose, ethyl cellulose, methyl hydroxypropyl cellulose, hydroxyethyl cellulose, sodium cellulose sulfate, hydroxypropyl cellulose, sodium carboxymethyl cellulose (CMC), crystalline cellulose and cellulose powder; and alginic acid-based polymers such as sodium alginate and propylene glycol esters of alginic acid.

The synthetic water-soluble polymers include ionic or non-ionic water-soluble polymers, for example, vinyl-based polymers such as polyvinyl alcohol, polyvinyl methyl ether, polyvinyl pyrrolidone and carboxyvinyl polymers (carbomers); acryl-based polymers such as sodium polyacrylate, poly ethyl acrylate, polyacrylamide compounds and acrylic acid/alkyl methacrylate copolymers (product name “pemulen TR-1”).

The polyacrylamide compounds particularly include polyacrylamide compounds consisting of homopolymers, copolymers or crosspolymers containing one or more constituent units chosen from among 2-acrylamido-2-methylpropane sulfonic acid (hereinafter sometimes abbreviated to “AMPS”), acrylic acid and derivatives thereof.

Specific examples of such polyacrylamide compounds include vinylpyrrolidone/2-acrylamido-2-methylpropane sulfonic acid (salt) copolymers, dimethylacrylarnide/2-acrylamido-2-methylpropane sulfonic acid (salt) copolymers, acrylamide/2-acrylamiclo-2-methylpropane sulfonic acid copolymers, dimethylacrylainide/2-acrylamido-2-methylpropane sulfonic acid crosspolymers crosslinked with methylenebisacrylamide, mixtures of polyacrylamide and sodium polyacrylate, sodium acrylate/2-acrylamido-2-methylpropane sulfonic acid copolymers, hydroxyethyl acrylate/2-acrylamido-2-methylpropane sulfonic acid (salt) copolymers, ammonium polyacrylate, polyacrylamide/ammonium acrylate copolymers, and acrylamide/sodium acrylate copolymers. However, the compounds are not limited to these examples.

Preferred examples of salts in the previous paragraph include alkali metal salts (such as calcium salts and magnesium salts), ammonium salts, organic amine salts (such as monoethanolamine salts, diethanolamine salts, and triethanolamine salts). One or more of these polyacrylamide compounds may be used.

These polyacrylamide compounds may be synthesized or obtained as commercial products. For example, the vinyl pyrrolidone/2-acrylarnido-2-methylpropane sulfonic acid (salt) copolymer may be “Aristoflex AVC” (manufactured by Clariant), the sodium alylate/2-acrylamido-2-methylpropane sulfonic acid (salt) copolymer may be “Simulgel EG” (manufactured by Seppic) or “Simulgel EPG” (manufactured by Seppic), the acrylamide/2-acrylamido-2-methylpropane sulfonic acid sodium salt copolymer may be “Simulgel 600” (manufactured by Seppic), the acrylamide/2-acrylamido-2-methylpropane sulfonic acid (salt) may be “Sepigel 305” (manufactured by Seppic) or “Sepigel 501” (manufactured by Seppic), the homopolymer of a 2-acrylamido-2-methylpropane sulfonic acid sodium salt may be “1-lostacerin AMPS” (manufactured by Clariant) or “Simulgel 800” (manufactured by Seppic), and the dimethylacrylamide/2-acrylamido-2-methylpropane sulfonic acid may be “SU-Polymer 0-1” (manufactured by Toho Chemical Industry). In the present invention, these polymers named Sepigel and Simulgel may be used together with polyacrylate crosspolymer-6.

The hydrophilic thickener in the water-based cosmetic may be a combination of one or more types.

### Emulsifiers

When the composition is in the form of an emulsion, it may contain an additional emulsifier. Emulsifiers may be nonionic, anionic, cationic, or zwitterionic.

### Other Optional Ingredients.

Compositions herein may include one or more optional ingredients known for use in topical cosmetic compositions, provided that the optional components do not unacceptably alter the desired benefits of the composition. In some instances, it may be desirable to select cosmetic actives that function via different biological pathways so that the actives do not interfere with one another. When the composition is in the form of an emulsion, the additional ingredients should not introduce instability into the emulsion (e.g., syneresis). For example, it may be desirable to select optional ingredients that do not form complexes with other ingredients in the composition, especially pH sensitive ingredients like vitamin B3 compounds, salicylates and peptides.

The additional ingredients should be suitable for use in contact with human skin tissue without undue toxicity, incompatibility, instability, allergic response, and the like. The optional components, when present, may be included at an amount of about 0.001% to 50% (e.g., 0.01% to 40%, 0.1% to 30%, 0.5% to 20%, or 1% to 10%), by weight of the composition. Some nonlimiting examples of additional ingredients include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunscreen agents, sunless tanning agents, lubricants, anti-acne agents, anti-cellulite agents, chelating agents, anti-wrinkle actives, anti-atrophy actives, phytosterols and/or plant hormones, N-acyl amino acid compounds, antimicrobials, and antifungals. Some particularly suitable examples of additional ingredient include one or more skin care actives selected from the group consisting of vitamin B3 compounds (e.g., niacinamide), n-acyl amino acids (e.g., undecylenoyl phenylalanine), vitamin E compounds (e.g., tocopheryl acetate), palmitoylated dipeptides (e.g., palmitoyl-lysine-threonine), palmitoylated pentapeptides (e.g., palmitoyl-lysine-threonine-threonine-lysine-serine), vitamin A compounds (e.g., retinol and retinyl propionate), and combinations thereof. Other non-limiting examples of optional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/0092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

Sucrose esters may be used herein. Such sucrose ester can be a blend of two or more sucrose esters, wherein the two or more sucrose esters are present at a ratio of any one sucrose ester to another of 1:10 to 1:1 (e.g., 1:7, 1:5, 1:3, or 1:2). In some instances, the sucrose ester may be a blend of sucrose laurate and sucrose dilaurate, wherein sucrose laurate is present at 50% to 80%, by weight of the sucrose ester, and the sucrose dilaurate is present at 20% to 45%, by weight of the sucrose ester. Alternatively, the sucrose ester may be a blend of sucrose laurate, sucrose dilaurate and sucrose trilaurate, wherein the sucrose dilaurate is present at 35% or more, by weight of the sucrose ester. A particularly suitable example of a sucrose ester for use herein is Sucrose Dilaurate BC10034 available from BASF.

### Method of Use of the Cosmetic Composition

The method of use herein includes identifying a target portion of skin on a person in need of treatment and applying the composition to the target portion of skin over the course of a treatment period. The target portion of skin may be on a facial skin surface such as the forehead, perioral, chin, periorbital, nose, and/or cheek) or another part of the body (e.g., hands, arms, legs, back, chest). The person in need of treatment is one whose skin exhibits signs of oxidative stress, such as fine lines, wrinkles, hyperpigmentation, uneven skin tone, and/or other visible skin features typically associated with aging. In some instances, the target portion of skin may not exhibit a visible sign of skin aging, but a user (e.g., a relatively young user) may still wish to target such an area of skin, if it is one that typically develops such issues as a person age. In this way, the present method may be used as a preventative measure to delay the onset of visible signs of skin aging.

The composition may be applied to a target portion of skin and, if desired, to the surrounding skin at least once a day, twice a day, or on a more frequent daily basis, during a treatment period. When applied twice daily, the first and second applications are separated by at least 1 to 12 hours. Typically, the composition is applied in the morning and/or in the evening before bed. The treatment period may last for at least 1 week (e.g., about 2 weeks, 4 weeks, 8 weeks, or even 12 weeks). In some instances, the treatment period will extend over multiple months (i.e., 3-12 months). In some instances, the composition may be applied most days of the week (e.g., at least 4, 5 or 6 days a week), at least once a day or even twice a day during a treatment period of at least 2 weeks, 4 weeks, 8 weeks, or 12 weeks.

The step of applying the composition may be accomplished by localized application. In reference to application of the composition, the terms “localized”, “local”, or “locally” mean that the composition is delivered to the targeted area (e.g., a wrinkle or line) while minimizing delivery to skin surfaces where treatment is not desired. The composition may be applied and lightly massaged into an area of skin. The form of the composition or the dermatologically acceptable carrier should be selected to facilitate localized application. While certain embodiments herein contemplate applying a composition locally to an area, it will be appreciated that compositions herein can be applied broadly to one or more skin surfaces. In certain embodiments, the compositions herein may be used as part of a multi-step beauty regimen, wherein the present composition may be applied before and/or after one or more other compositions.

### Combinations

1. A cosmetic composition comprising by weight:
(a) from about 1.5% to about 5% of polyacrylate crosspolymer-6;
(b) from about 0.05% to about 5% of a nonionic surfactant having HLB of from about 10 to about 20; and
(c) aqueous carrier,
wherein the composition meets at least one of the following conditions (i)-(iii):
(i) wherein the pH of the composition is between about 2.0 and about 5.0;
(ii) wherein the nonionic surfactant is liquid at 20° C.; and
(iii) wherein the composition is substantially free of hydroxyethylcellulose and xanthan gum.
2. The composition of the preceding feature, wherein the composition comprises from about 1.8% to about 4%, preferably from about 2% to about 4% of polyacrylate crosspolymer-6.
3. The composition of any of the preceding features, wherein the weight ratio between the polyacrylate crosspolymer-6 and the nonionic surfactant is from about 15:1 to about 1:15, preferably from about 8:1 to about 1:8, alternatively from about 5:1 to about 1:5, alternatively from about 5:1 to about 1:3.

### EXAMPLES

### Cosmetic Compositions (Wt %)

| Ingredient Name | Ex. 1 | Ex. 2 | Ex. 3 | Ex. 4 | Ex. 5 | Ex. 6 | CEx. i | CEx. ii |
|---|---|---|---|---|---|---|---|---|
| Water | qs | qs | qs | qs | qs | qs | qs | qs |
| D- Panthenol | 0.5 | 0.5 | 0.5 | 0.5 | 0.5 | 0.5 | 0.5 | 0.5 |
| Niacinamide | 2.0 | 2.0 | 2.0 | 2.0 | 2.0 | 2.0 | 2.0 | 2.0 |
| Xylitol | 1.0 | 1.0 | 1.0 | 1.0 | 1.0 | 1.0 | 1.0 | 1.0 |
| pH buffer | 3.1 | 3.1 | 3.1 | 3.1 | 3.1 | 3.1 | 3.1 | 3.1 |
| Preservative | 0.15 | 0.15 | 0.15 | 0.15 | 0.15 | 0.15 | 0.15 | 0.15 |
| Sepimax Zen *1 | 2.4 | 2.4 | 2.4 | 2.1 | 2.0 | 2.4 | 2.4 | 2.4 |
| Sepigel 305 *2 | 0.5 | 0.5 | 0.5 | 0.5 | 0.5 | 0.5 | 0.5 | 0.5 |
| Dimethicone 5 cst | 1.75 | 1.75 | 1.75 | 1.75 | 1.75 | 1.75 | 1.75 | 1.75 |
| Dimethicone 50 cst | 1.75 | 1.75 | 1.75 | 1.75 | 1.75 | 1.75 | 1.75 | 1.75 |
| DC1503 Fluid *3 | 1.5 | 1.5 | 1.5 | 1.5 | 1.5 | 1.5 | 1.5 | 1.5 |
| Glycereth-25 PCA | 0.6 | — | — | — | — | — | — | — |
| Isostearate (HLB = 14) | | | | | | | | |
| Polysorbate 20 | 0.25 | — | — | 0.1 | 2.0 | 0.24 | — | — |
| (HLB = 16.7) | | | | | | | | |
| Laureth-7 | — | 0.85 | — | — | — | — | — | — |
| (HLB = 12.3) | | | | | | | | |
| PEG-100 Stearate | — | — | 0.85 | — | — | — | — | — |
| (HLB = 18.8) | | | | | | | | |
| Laureth-4 (HLB = 9.7) | — | — | — | — | — | — | — | 0.85 |
| Glycerin | 5.5 | 5.5 | 5.5 | 5.5 | 5.5 | 5.5 | 5.5 | 5.5 |
| Pentylene Glycol | 0.02 | 0.02 | 0.02 | 0.02 | 0.02 | 0.02 | 0.02 | 0.02 |
| Amihope LL *4 | 2.0 | 2.0 | 2.0 | 2.0 | 2.0 | 2.0 | 2.0 | 2.0 |
| Goddball G6C *5 | 1.5 | 1.5 | 1.5 | 1.5 | 1.5 | 1.5 | 1.5 | 1.5 |
| Fragrance | 0.04 | 0.04 | 0.04 | 0.04 | 0.04 | 0.04 | 0.04 | 0.04 |
| pH of the composition | 3.78 | 3.87 | 3.84 | 3.81 | 3.81 | 3.81 | 3.86 | 3.84 |
| Smooth Flowability | 42.2 | 59.7 | 52.7 | 15.6 | 51.1 | 24.4 | Control | 16.0 |
| when tilted/shaken | | | | | | | | |
| Ease of taking the | 38 | 50 | 42 | 20 | 47 | 40 | Control | 0.4 |
| right amount from | | | | | | | | |
| package | | | | | | | | |

### Details of the Ingredients Used in the Above Table

| | | |
|---|---|---|
| *1 | Sepimax Zen | INCI: Polyacrylate crosspolymer-6 |
| *2 | Sepigel 305 | Containing 40% of Polyacrylamide as active. INCI: |
| | | Polyacrylamide & Water & |
| | | C13-14 Isoparaffin & Lauleth-7 |
| *3 | DC1503 | Dimethicone (5 cSt, Solvent) and Dimethiconol |
| *4 | Amihope LL | Lauroyl lysin available from |
| | | Ajinomoto, having a flat hexagonal |
| | | shape and having a mean |
| | | particle size of 20-30 microns |
| *5 | Goddball G6C | Silica having a mean particle |
| | | size of 3-5 microns and an oil |
| | | absorbance of 30 ml/100 g, |

### Evaluation of Flowability of the Composition

### Flowability of the Composition are Evaluated by the Following Method:

10 panelists assessed the products (blinded) filled in 80 g glass jar (consistent weight of product and type of jars). Then the panelists evaluated “the ease of getting the right amount from the package” and “smooth flowability when tilted/shaken” and gave a score between 1 and 5, 1 being excellent, 5 being poor. Average scores of 10 panelists were obtained for each example compositions. Then, the average score of one example composition was compared to the average score of CEx.i as Control. % improvement of the score compared to Control is described above in the table.

Ex. 1 through Ex. 6 are the inventive examples, which are suitably used as oil-in-water emulsion cosmetic composition, in a form of essence, lotion, serum and/or gel cream. CEx. i and CEx.ii are comparative examples. CEx.i does not contain any nonionic surfactant, and CEx. ii contains lower HLB nonionic surfactant. The inventive examples can provide improved flowability of the composition, compared to the comparative examples.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A cosmetic composition comprising by weight:
(a) from about 1.5% to about 5% of polyacrylate crosspolymer-6;
(b) from about 0.05% to about 5% of a nonionic surfactant chosen from PEG-100 stearate, laureth-7, polysorbate 20, glycereth-25 PCA isostearate, or mixtures thereof;
(c) an aqueous carrier;
wherein the composition comprises a pH from about 2.0 to about 5.0; and
wherein the composition is substantially free of hydroxyethylcellulose and xanthan gum.

2. The composition of claim 1, wherein the composition comprises from about 1.8% to about 4% of polyacrylate crosspolymer-6.

3. The composition of claim 2, wherein the composition comprises from about 2% to about 4% of polyacrylate crosspolymer-6.

4. The composition of claim 1, wherein the weight ratio between the polyacrylate crosspolymer-6 and the nonionic surfactant is from about 15:1 to about 1:15.

5. The composition of claim 4, wherein the weight ratio between the polyacrylate crosspolymer-6 and the nonionic surfactant is from about 8:1 to about 1:8.

6. The composition of claim 5, wherein the weight ratio between the polyacrylate crosspolymer-6 and the nonionic surfactant is from about 5:1 to about 1:5.

7. The composition of claim 6, wherein the weight ratio between the polyacrylate crosspolymer-6 and the nonionic surfactant is from about 5:1 to about 1:3.

8-14. (canceled)
